(19) United States
(12) Patent Application Publication
(10) Pub. No.: US 2013/0097781 A1
(43) Pub. Date: Apr. 25, 2013

(54) DEVICE AND METHOD FOR STABILIZATION OF ANGIOGRAPHY TABLE

(71) Applicant: Jeffrey E. Thomas, Hillsborough, CA (US)

(72) Inventor: Jeffrey E. Thomas, Hillsborough, CA (US)

(73) Assignee: BRAINSTORM TECHNOLOGIES, INC., San Francisco, CA (US)

(21) Appl. No.: 13/695,908

(22) PCT Filed: May 5, 2011
(86) PCT No.: PCT/US11/35392

§ 371 Date: Dec. 19, 2012

Related U.S. Application Data
(60) Provisional application No. 61/331,528, filed on May 5, 2010.

(51) Int. Cl. A61B 6/04

(52) U.S. Cl. CPC A61B 6/0407
USPC 5/601

(57) ABSTRACT

A stabilization device includes a base, a column, and a table support structure, wherein the column is disposed within the base and is movable within the base along a vertical axis. The table support structure is connected to the column and comprises a substantially planar top surface. The stabilization device may be positioned under an extension of a patient support table and adjusted along the vertical axis so that a substantially planar top surface of the table support structure is disposed flush against a substantially planar bottom surface of the extension.

This application claims priority to International Application No. PCT/US2011/035392, filed on May 5, 2011, which claims priority to U.S. Provisional Application No. 61/331,528, filed on May 5, 2010.

### BACKGROUND

This application relates in general to a device for use in stabilizing a patient support table during surgery. Specifically, the application relates to the use of a device and method for stabilizing an angiography table.

Generally, operating environments are either equipped for neurointerventional radiology or micro-surgical for vascular brain surgery. That is, either the operating environment is equipped to provide equipment suitable of interventional angiography techniques or open micro-surgical techniques. However, rarely are operating rooms equipped with both types of equipment. It may be preferable to provide an operating environment where multiple treatment modalities for cerebrovascular disease like stroke and cerebral aneurysms can be combined or used sequentially without displacing the patient.

As such, patient support tables that may be used for neurointerventional radiology must be adapted for use during a micro-surgical procedure by, for example, adding an extension to the end of the table. This extension supports the patient's body and allows a radiolucent headrest to be attached to the end of the extension to support the patient's head in the air during surgery. This extension may not be supported or stabilized vertically beyond being attached to the original table along the horizontal plane. Therefore, it may be advantageous to provide a device that would easily allow the surgeon or operating team to provide additional support to the table extension along the vertical plane.

### BRIEF DESCRIPTION OF DRAWINGS

The accompanying drawings, which are incorporated in and constitute a part of the specification, illustrate various example systems, methods, and so on, that illustrate various example embodiments of aspects of the invention. It will be appreciated that the illustrated element boundaries (e.g., boxes, groups of boxes, or other shapes) in the figures represent one example of the boundaries. One of ordinary skill in the art will appreciate that one element may be designed as multiple elements or that multiple elements may be designed as one element. An element shown as an internal component of another element may be implemented as an external component and vice versa. Furthermore, elements may not be drawn to scale.

FIG. 1 is a side view of a patient support table stabilization device.

FIG. 2 is a side view of a patient support table stabilization device in use with a patient support table.

FIG. 3 is a side view of a patient support table stabilization device in use with a patient support table.

FIG. 4 is a side view of a patient table stabilization device in use with a patient support table.

### DETAILED DESCRIPTION

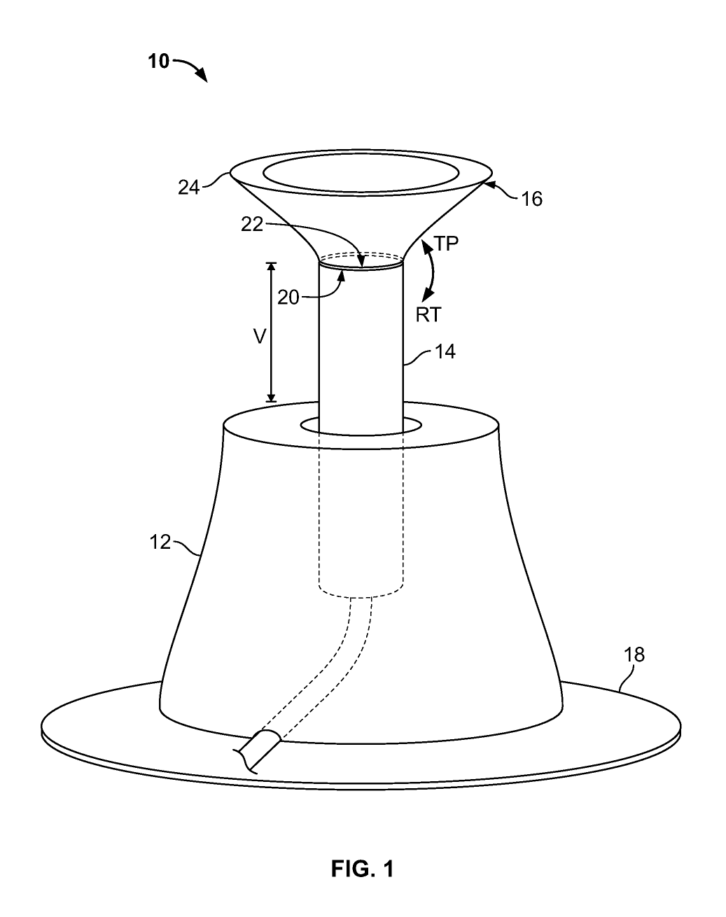

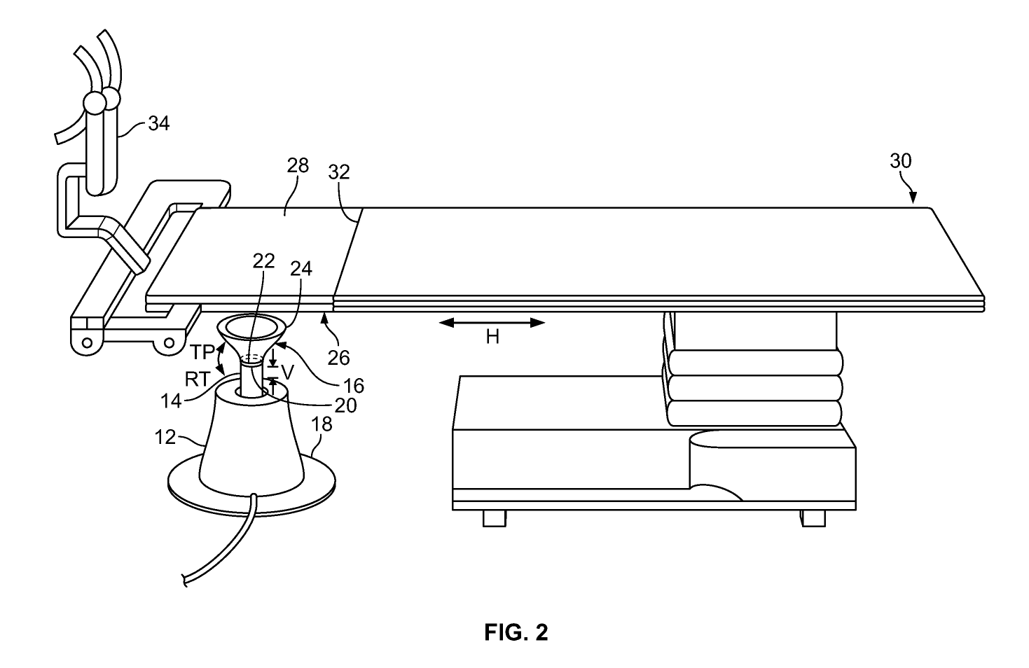

As shown in FIG. 1, a stabilization device 10 for stabilizing a patient support table 30 is provided. Referring to FIG. 2, some patient support tables 30, such as the Allura AD7 table available from Philips Healthcare, may include an extension 28 that extends horizontally from the proximal side 32 of the patient support table 30. During neurovascular surgery, it may be necessary to fit the patient support table 30 with a radiolucent headrest 34 to stabilize the patient's head. For some procedures, the radiolucent headrest 34 is attached to the end of the extension 28 and the patient's body is moved onto the extension 28 so that the physician may conduct 360° radiological scans of the patient's head while the patient is stabilized within the headrest 34 and positioned largely on the extension 28. As shown in FIG. 2, the extension 28 may not be supported or stabilized vertically beyond being attached to the proximal side 32 of the patient table 30 along the horizontal plane H. The stabilization device 10 may be placed under the extension 28 and used to stabilize the patient during these procedures.

Referring again to FIGS. 1 and 2, in one embodiment, the device includes a base 12, a column 14, and a table support structure 16. The base 12 may include a floor stand 18 to support the stabilization device 10. The floor stand 18 may include retractable wheels (not shown) in order to permit the stabilization device 10 to be moved easily about the operating room.

The column 14 may be disposed within or at the top of the base 12. As shown in FIG. 1, when the base 12 is positioned on the floor of the operating room, the column 14 is generally perpendicular to the floor, along a vertical axis V. The column 14 moves along the vertical axis within the base 12 to permit the table support structure 16 to be raised and lowered.

The base 12 may be of any suitable shape and may be used the house the control mechanism used to move the table support structure 16, disposed at the upper end 22 of the column 14, up and down relative to the floor of the operating room. Suitable control mechanisms and operators (not shown) known in the art which are capable of raising, lowering or tilting the table support member 16 are contemplated. The stabilization device 10 may include control mechanisms or operators that move the column 14 within the base 12 using hydraulics, pistons, pneumatics, electric, air or other known automated methods. Alternatively, the column 14 may be manually movable within the base 12 so that the height of the column 14 may be adjusted without the use of electricity. In one embodiment, the column 14 may be adjusted with a remote device (not shown), such as a wired or wireless remote control or a wired control panel affixed to the wall of the operating room.

Referring again to FIG. 1, the table support structure 16 may generally be frusto-conical with a substantially flat, substantially planar top surface 24 having a larger diameter than the substantially flat bottom surface 20; however other shapes and dimensions are contemplated for the table support structure 166 and its top 24 and bottom surfaces 20. The table support structure 16 may be made of radiolucent material, such as carbon fiber composite or plastic that does not interfere with angiograph or other scanning procedures. Other known suitable radiolucent and non-radiolucent materials, such as rubber may be used to make the table support structure 16. In yet another embodiment, the table support structure 16 may be made of a non-radiolucent material.

As shown in FIG. 2, the substantially planar top surface 24 is positioned so that it contacts the bottom surface 26 of the extension 28 of the patient support table 30, thereby stabilizing the extension 28. The substantially planar top surface 24 may be positioned flush against the generally planar bottom surface 26 of the extension 28, supporting the weight of the patient (not shown) positioned on the extension 28.

The table support structure 16 is connected to the base 12 of the stabilization device 10 with the column 14. In one embodiment, the bottom surface 20 of the table support structure 16 is attached to the upper end 22 of the column 14. The table support structure 16 may be rotatably or pivotably connected to the upper end 22 of the column 14 so that the table support structure 16 can move with the patient support table 30 into a Trendelenberg position TP or a reverse Trendelenberg position RT or tilted from side to side, as if to roll the patient. The tilt function is provided to allow the table support structure 16 to be moved when the patient support table 30 is tilted, leaving the column 14 and/or the base 12 stationary.

In one embodiment, the table support structure 16 may be connected to the upper end 22 of the column 14 with a ball bearing or U-joint (not shown) or other mechanisms suitable to allow the table support structure 16 to be tilted up and down or rolled from side to side. Optionally, the table support structure 16 may be locked into a position by a locking mechanism (not shown), such as with the use of pins, screws, bolts, or other suitable means. The weight of the patient and the contact with the patient support table 30 may be sufficient to maintain proper stability of the patient support structure during surgical procedures.

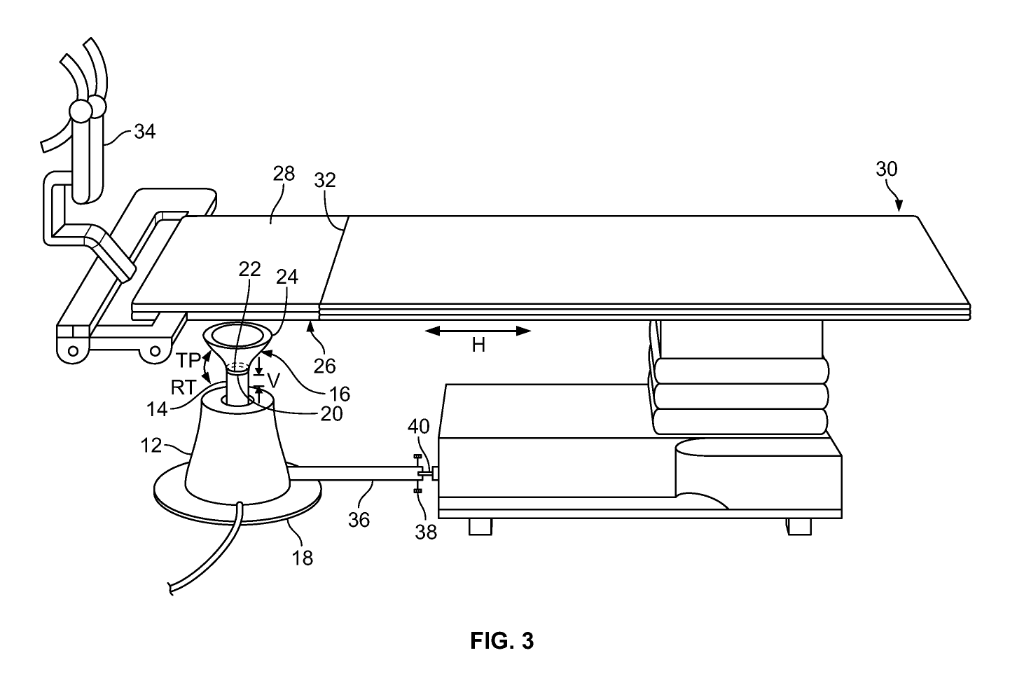

The stabilization device 10 may be separate from or connected to the patient support table 30. As shown in FIG. 3, in one embodiment, the stabilization device 10 is connected to the support table 30. In this embodiment, the stabilization device 10 includes a connectable arm 36 disposed toward the base 12 or the floor stand 18 of the stabilization device 10. The connectable arm 36 includes at least one bolt 38 or other suitable connecting mechanism that allows the arm 36 to be securely fastened to the bottom of the patient support table 30 to provide further stability.

The device may be connected to the patient support table 30 by moving the connectable arm 36 of the stabilization device 10 to a connection pin 40 located at the bottom of the patient support table 30. The arm 36 may then be securely fastened to the bottom of the patient support table 30 by tightening the bolt or bolts 38 securely to the connection pin 40.

Referring again to FIG. 2, a method for stabilizing a patient support table is also provided. The method includes providing a patient support table 30 for use in neurointerventional radiology. An extension 28 of the patient support table 30 may be extended horizontally H so that after a scanning process or a neurointerventional procedure is complete, the patient can be moved to a position on the patient support table 30 that would allow the patient to have an operative neurological procedure executed. In order to execute a surgical procedure, the patient is moved so that their head extends past the end of the extension 28 and is placed in a headrest 34.

The method also includes providing a stabilization device 10, as described above, that includes a base 12, a column 14, and a table support structure 16. The stabilization device 10 is positioned under the extension 28 and the column 14 is moved along the vertical axis V so that the substantially planar top surface 24 of the table support structure 16 is disposed flush against a substantially planar bottom surface 26 of the extension 28. The column 14 and table support structure 16 may be remotely moved into place under the extension 28 with a wireless remote device or wired control panel for the operating room. The patient support table 30 and the extension 28 may be tilted in a Trendelenberg, reverse Trendelenberg position, or other desired position, using the control mechanisms provided with the patient support table 30. Preferably, the table support structure 16 does not need to be separately adjusted. Rather, the table support structure 16, supporting the weight of the patient, will move with the extension 28 and the patient support table 30.

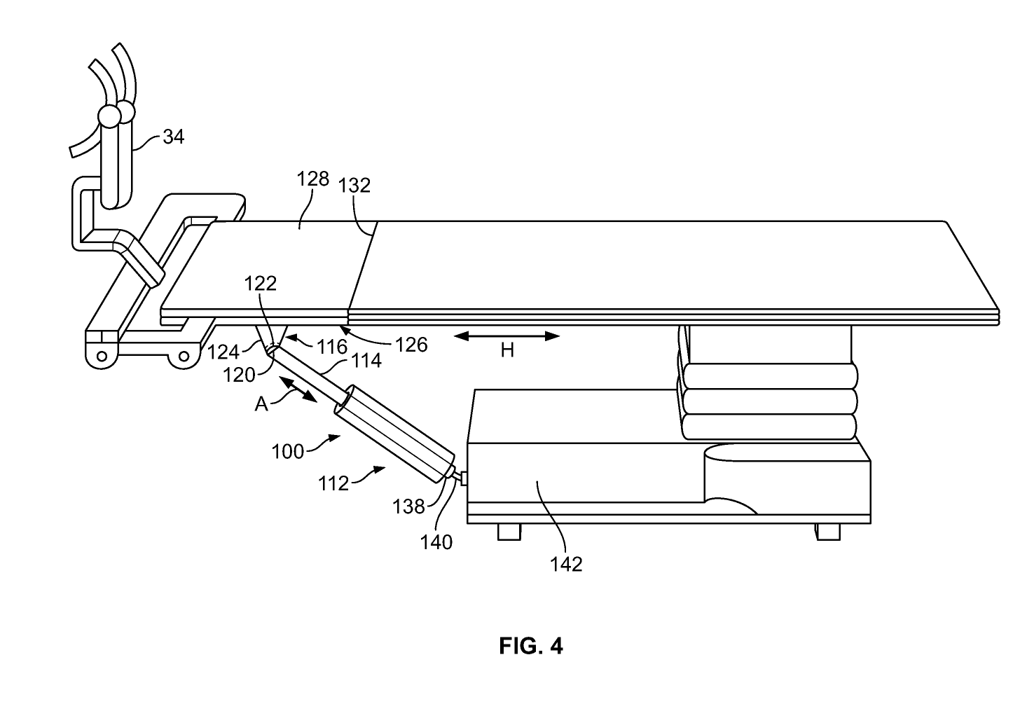

Referring to FIG. 4, another embodiment of the stabilization device 100 includes a base 112, a column 114, and a table support structure 116. In this embodiment, the base 112 is attached to the bottom 132 of the patient support table 130 instead of to a floor stand, as described above. By mounting the base 112 to the bottom 142 of the patient support table 130, the stabilization device 100 can be moved with the table 130, instead of independently from the table 130. The base 112 may include at least one bolt 138 or other suitable connecting mechanism that will allow the base 112 to be securely fastened to the bottom 142 of the patient support table 130. The base 112 may then be securely fastened to the bottom 142 of the patient support table 130 by tightening a bolt or bolts 138 on the base 112 securely to a connection pin 140 on the table 130. It is contemplated that other suitable connection mechanisms may be appropriate.

Again referring to FIG. 4, the column 114 is disposed within the base 112 along an axis A to permit the table support structure 116 to be raised and lowered, relative to the base 112. The stabilization device 100 may generally include control mechanisms and operators (not shown) known in the art suitable to raise or tilt the table support member 116 relative to a patient support table.

The stabilization device 100 may include control mechanisms or operators that move the column 114 within the base 112 using hydraulics, pneumatics, electric, air or other known automated methods. Alternatively, the column 114 may be manually movable within the base 112 so that the height of the column 114 may be adjusted without the use of electricity. In another embodiment, the column 114 may also be adjusted with a remote device (not shown), such as a wired or wireless remote control or a wired control panel affixed to the wall of the operating room.

The stabilization device 100 also includes a table support structure 116 with a bottom surface 120 that is attached to the upper end 122 of the column 114. As shown in FIG. 4, the table support structure 116 also includes a substantially planar top surface 124 that may be moved into contact with the bottom surface 126 of the extension 128 of the patient support table 130, thereby stabilizing the extension 128. The substantially planar top surface 124 may be positioned flush against the generally planar bottom surface 126 of the extension 128, supporting the weight of the patient (not shown) positioned on the extension 128.

The table support structure 116 may be rotatably or pivotably connected to the upper end 122 of the column 114 so that the table support structure 116 can move with the patient support table 130 into a Trendelenberg position TP or a reverse Trendelenberg position RT. This tilt function is provided to allow the table support structure 116 to be pivoted relative to the base 112. In one embodiment, the table support structure 116 may be connected to the column 114 with a ball bearing or U-joint (not shown) or other mechanisms suitable to allow the table support structure 116 to be tilted. Optionally, the table support structure 116 may be locked into a position by a locking mechanism (not shown), but generally, the weight of the patient and the connection to the patient support table will be sufficient to maintain proper stability during surgical procedures.

Referring again to FIG. 4, the table support structure 116 may generally be frusto-conical with a substantially flat, substantially planar top surface 124 having a larger diameter than the substantially flat bottom surface 120. In another embodiment, the table support structure 116 will be made of radiolucent material, such as carbon fiber composite or plastic. Other known suitable radiolucent and non-radiolucent materials, such as rubber may be used to make the table support structure 116.

While example methods and compositions have been illustrated by describing examples, and while the examples have been described in considerable detail, it is not the intention of the applicant to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention is not limited to the specific details, the representative apparatus, and illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

1. A stabilization device for use with a patient support table, comprising:
a base;
a column, wherein the column is disposed within the base and is movable within the base along a vertical axis; and
a table support structure, wherein the table support structure is pivotably connected to an upper end of the column and comprises a substantially planar top surface.

2. The stabilization device of claim 1, wherein the table support structure is pivotably connected to the column so that the table support structure can move with the patient support table into a trendelenberg position or a reverse trendelenberg position.

3. The stabilization device of claim 1, wherein the column is hydraulically, electrically, pneumatically, or manually activated.

4. The stabilization device of claim 3, wherein the column is hydraulically activated.

5. The stabilization device of claim 4, wherein the column may be activated with a remote device.

6. The stabilization device of claim 1, wherein the table support structure comprises radiolucent material.

7. The stabilization device of claim 1, wherein the table support structure comprises of rubber, plastic, or carbon fiber composite.

8. The stabilization device of claim 1, wherein the table support structure comprises radiolucent plastic material.

9. The stabilization device of claim 1, wherein the stabilization device comprises a floor stand.

10. The stabilization device of claim 1, wherein the base of the stabilization device is configured to attached to the patient support table.

11. A method for stabilizing a patient support table for use in neurointerventional radiology, wherein the patient support table includes an extension capable of supporting a patient that is moved on to the extension so that the patient's head may extend past an end of the extension, comprising:
providing a stabilization device, the stabilization device comprising a base, a column disposed and movable within the base, and a table support structure comprising a substantially planar top surface and a bottom surface, wherein the bottom surface is connected to an upper end of the column;
wherein the table support structure is capable of being positioned under the extension; and
wherein the column is configured to be adjusted to position the substantially planar top surface of the table support structure against a substantially planar bottom surface of the extension.

12. The method of claim 10, wherein the table support structure is pivotably connected to the column.

13. The method of claim 11, wherein the table support structure further includes a locking mechanism.

14. The method of claim 10, wherein the column is hydraulically, electrically, pneumatically, or manually activated.

15. The method of claim 13, wherein the column is hydraulically activated.

16. The method of claim 14, wherein the column may be controlled with a remote device.

17. The method of claim 10, wherein the table support structure comprises radiolucent material.

18. The method of claim 10, wherein the table support structure is comprises of rubber, plastic, or carbon fiber composite.

19. The method of claim 10, wherein the table support structure comprises radiolucent plastic material.

20. The method of claim 10, further comprising the step of attaching the base of the stabilization device to the patient support table.
